(19) United States
(12) Patent Application Publication
(10) Pub. No.: US 2022/0362499 A1
(43) Pub. Date: Nov. 17, 2022

(54) A LARYNGOSCOPE FOR OROTRACHEAL INTUBATION

(71) Applicant: MEDSNIPER S.R.L., Savona (IT)

(72) Inventor: Andrea MAZZA, Savona (IT)

(21) Appl. No.: 17/770,731

(22) PCT Filed: Oct. 16, 2020
(86) PCT No.: PCT/IB2020/059757

§ 371 Date: Apr. 21, 2022

(30) Foreign Application Priority Data
Oct. 22, 2019 (IT) 102019000019562

(51) Int. Cl. A61M 16/04; A61B 1/267; A61B 1/005; A61B 1/015

(52) U.S. Cl. CPC A61M 16/0488; A61B 1/267; A61B 1/0051; A61B 1/015; A61B 1/00045

(57) ABSTRACT

A laryngoscope for orotracheal intubation, includes a tubular body that serves as a guide for an endotracheal tube. The tubular body has an adjustable head which is inserted into the pharynx through the mouth following the anatomical curvature until it reaches the larynx and vocal cords. The head may be equipped with a lighting and display system that can be connected to an external screen. The head can be oriented by means of actuators so that it can be directed towards the entrance to the airways,

### FIELD OF THE INVENTION

The present invention relates in general to the field of medical devices.

More specifically, the invention relates to a laryngoscope for orotracheal intubation.

### DESCRIPTION OF THE PRIOR ART

Orotracheal intubation consists of placing an endotracheal tube inside the airways to allow mechanical or manual ventilation of the patient and to protect the lungs from reflux of gastric material or material coming from the upper airways.

In the current state of the art, the endotracheal tube is placed in a patient's trachea by means of one of the following operations:

direct laryngoscopy: a laryngoscope (e.g. a Macintosh laryngoscope) is used to lift the anatomical structures (pharynx, larynx) and to allow visualization of the vocal cords and access to the airways for insertion of the endotracheal tube; direct laryngoscopy may cause edema (therefore swelling) and block the airways; direct laryngoscopy intubation must be performed by a particularly experienced doctor (typically an anesthesiologist) and requires special preparation of the patient;

videolaryngoscopy: involves the use of laryngoscopes with remote vision systems, which allow the doctor performing the operation to view the deeper anatomical parts on an external screen; this technique has simplified and made intubation performed by direct laryngoscopy safer and less traumatic, but is still affected by many of the limitations of intubation performed by direct laryngoscopy;

laryngoscopy with a flexible fiberscope: this operation involves inserting a fiberscope into the airways and using the fiberscope as a guide on which the endotracheal tube is slid to obtain intubation; this operation is very safe and overcomes the limits of direct laryngoscopy, but requires very experienced personnel and expensive devices (fiberscope) that are not always available.

### OBJECT AND SUMMARY OF THE INVENTION

The present invention aims to provide a laryngoscope for orotracheal intubation that overcomes the problems of the prior art.

According to the present invention, this object is achieved by a laryngoscope for orotracheal intubation having the characteristics forming the subject of claim 1.

The claims form an integral part of the disclosure provided here in relation to the invention.

As will become clear in the course of the detailed description that follows, the laryngoscope according to the present invention comprises a tubular body having an orientable head which is inserted into the pharynx through the mouth following the anatomical curvature until it reaches the larynx and the vocal cords. The head may be equipped with a lighting and display system (camera) that can be connected to an external screen. The head can be oriented by means of actuators so that it can be directed towards the entrance to the airways. Once the airways have been visualized and aligned, the endotracheal tube is inserted up past them, using the tubular body of the laryngoscope as a guide.

The tubular body of the laryngoscope can be provided with a movable element placed on the front wall of the head to allow lifting of the epiglottis in cases where the epiglottis obstructs vision and access to the vocal cords.

The tubular body can be provided with channels for fluids, for example, for delivering gas or drugs or for the suction of secretions, or a channel for the cables of a vision system.

The tubular body of the laryngoscope according to the present invention, thanks to the directional head, allows many of the difficulties connected with orotracheal intubation to be overcome, and is particularly effective in cases of difficult intubation or intubation in a hostile environment, where the conditions for a normal laryngoscopy do not exist.

### BRIEF DESCRIPTION OF THE DRAWINGS

The present invention will now be described in detail with reference to the attached drawings, given purely by way of non-limiting example, wherein:

FIG. 1 is a schematic view of a system for orotracheal intubation using a laryngoscope according to the present invention,

FIG. 2 is a partial schematic side view of the laryngoscope of FIG. 1,

FIG. 3 is a schematic cross-section along the line III-III of FIG. 2,

FIG. 4 is a schematic view of the detail indicated by the arrow IV in FIG. 2,

FIG. 5 is a side view according to the arrow V of FIG. 2,

FIG. 6 is a schematic view illustrating a laryngoscope according to the present invention inserted in the trachea of a patient,

FIGS. 7 and 8 are side views illustrating the main characteristic dimensions of a laryngoscope according to the present invention,

FIGS. 9 and 10 are cross-sections along the lines IX-IX and X-X of FIG. 7 illustrating the main characteristic dimensions of a laryngoscope according to the present invention, and

FIG. 11 is a front view illustrating the main characteristic dimensions of the element indicated by the arrow XI in FIG. 7.

### DETAILED DESCRIPTION

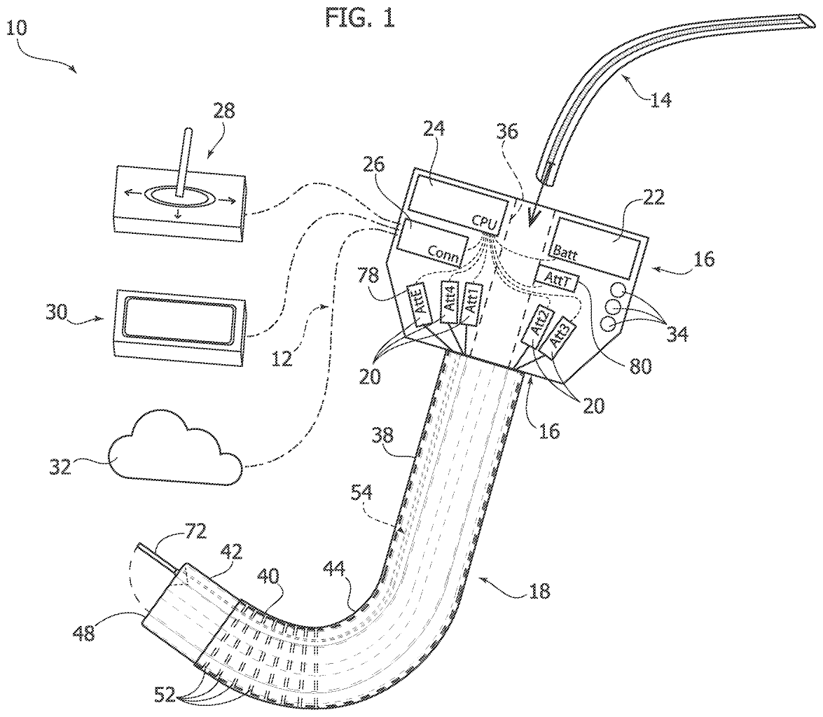

With reference to FIG. 1, numeral 10 indicates—in its entirety—a system for orotracheal intubation of patients. The system 10 includes a laryngoscope 12 configured to be inserted into a patient's trachea by passing through the mouth and larynx. The laryngoscope is configured to guide the insertion of an endotracheal tube 14.

The laryngoscope 12 comprises a base 16 and a tubular body 18 having a general J shape. The tubular body 18 may be removably fixed to the base 16.

The base 16 carries a plurality of actuators 20 therein. The actuators may be electric actuators powered by a battery 22 and controlled by an electronic control unit 24. Alternatively, the actuators 20 may be mechanical actuators.

The electronic control unit 24 may be connected to an interface unit 26 connected to a movement control device 28 external to the base 16, consisting, for example, of a joystick. The interface unit 26 may also be connected to a display 30. The connection between the interface unit 26 and the movement control device 28 and the display 30 may be made using cables.

In a possible embodiment, the interface unit 26 may be provided with a wireless communication protocol, which allows connection to a wireless communication network 32 that may consist of the Internet. In this case, the movement control system 28 and the display 30 may be connected to the interface unit 26 and to the electronic control unit 24 via the wireless communication network 32.

The base 16 of the laryngoscope 12 may comprise a plurality of fluid connectors 34. The base 16 may be provided with a through-hole 36 configured for the passage of the endotracheal tube 14.

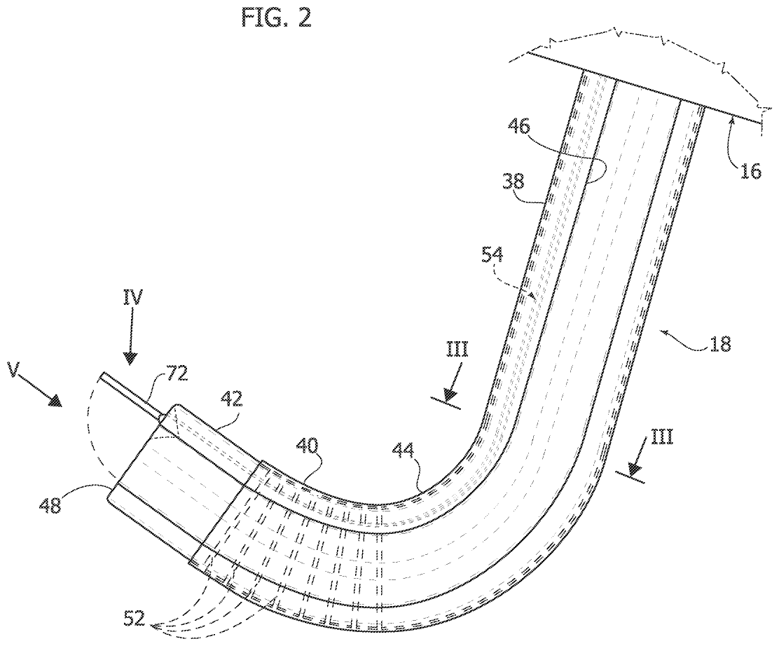

With reference to FIG. 2, the tubular body 18 comprises a proximal portion 38, a deformable section 40 and a head 42. The proximal portion 38 can be fixed to the base 16 in a releasable way. The proximal portion 38 may have a straight tract adjacent to the base 16 and can be connected to the deformable section 40 by means of a non-deformable curved tract 44.

The tubular body 18 has a central channel 46 that extends continuously between the opposite ends of the tubular body 18. At the proximal end of the tubular body 18, the central channel 46 communicates with the through-hole 36 of the base 16 and, at the opposite end, is open on a front surface 48 of the head 42. The central channel 46 of the tubular body 18 is configured to receive and guide the endotracheal tube 14.

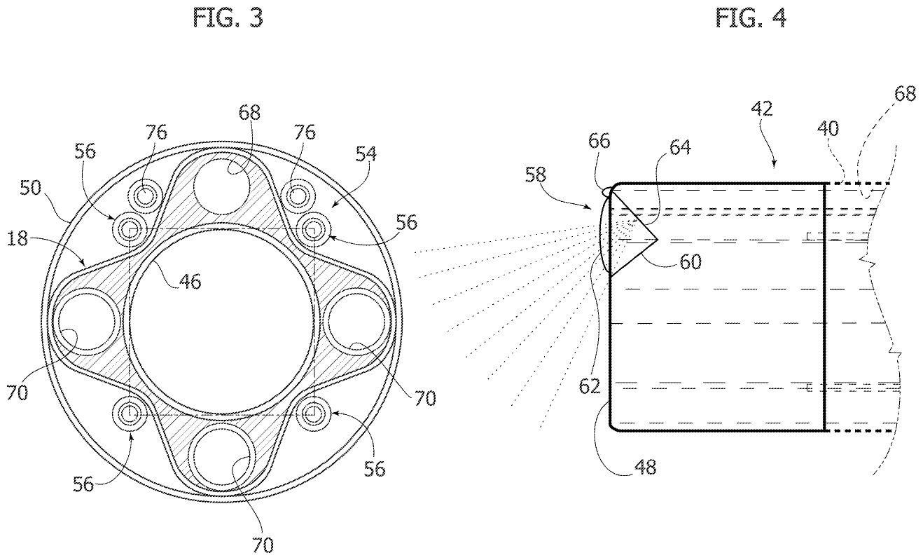

With reference to FIG. 3, in a cross-section, the tubular body 18 may have an outer profile with a plurality of lobes and depressions. The central channel 46 may have a circular cross-section. The tubular body may have an outer covering 50 formed by a thin-walled tubular sheath.

The deformable section 40 of the tubular body 18 may have a plurality of transverse cuts 52 (FIG. 2), which reduce the full cross-section of the tubular body and form weakening areas, which allow elastic bending of the deformable section 40. The elastic deformation of the deformable section 40 allows an inclination of the head 42 in any plane passing through the longitudinal axis of the head 42.

The head 42 is connected to the actuators 20 located in the base 16 by means of a transmission system 54 that extends along the tubular body 18. In a possible embodiment, the transmission system 54 comprises a plurality of Bowden cables 56, each of which comprises a guide sheath inside which a sliding cable is housed. The inner cables of the Bowden cables 56 are anchored to the head 42 at their distal ends. Each actuator 20 of the base 16 is associated with a respective Bowden cable 56. The actuators 20, controlled by means of the movement control device 28, are configured to control a longitudinal movement of the cable inside the respective sheath. The axial movement of the cables controlled by the actuators 20 changes the inclination of the head 42 due to an elastic deformation of the deformable section 40.

With reference to FIG. 3, the Bowden cables 56 that control the inclination of the head 42 may be arranged within respective depressions of the outer lobe profile of the tubular body 18. In a possible embodiment, four Bowden cables 56 may be provided, arranged at the vertices of a square whose center coincides with the center of gravity of the tubular body 18. The tensioning of the cables 56 produces a deformation of the deformable section 40 only. For this object, the material forming the proximal portion 38 and the head 42 may be a semi-rigid plastic material. The elastic deformability of the deformable section 40 may be obtained thanks to the deformability of the material as well as the weakening of the section generated by the transverse cuts 52.

With reference to FIG. 3, the simultaneous tensioning of two cables 56 located along the same side of the square produces an inclination of the head 42 in the direction of the side on which the tensioned cables are located. For example, a simultaneous tensioning of the two cables located along the upper horizontal side of the square produces an upward inclination of the head 42; the tensioning of the two cables 56 located along the right side of the square creates an inclination towards the right of the head 42, etc. The tensioning of a single cable 56 creates an inclination of the head 42 along a diagonal of the square. For example, the tensioning of the cable 56 located on the upper right corner of the square causes an inclination of the head upwards and to the right; the tensioning of the cable 56 located at the lower left causes an inclination of the head 42 downwards and to the left, etc.

With reference to FIG. 4, the laryngoscope 12 comprises a vision system 58 arranged on the front surface 48 of the head 42. The vision system 58 comprises an optical sensor 60, for example a CCD sensor, a lens 62 and a mirror 64. The lens 62 focuses the field of vision on the mirror 64, and the mirror 64 reflects the images onto the optical sensor 60. This arrangement makes it possible to direct the front field of view in an area comprised between the center of the central channel 46 up to about 5-6 cm forward with respect to the front wall 48 of the head 42. The vision device 58 may also comprise a light radiation source 66, for example an LED, to illuminate the field of view of the vision device 58. The optical system 60 and the light radiation source 66 are connected to the base 16 by means of cables which can extend inside a channel 68 formed in the tubular body 18. The images detected by the optical sensor 60 may be processed by the electronic control unit 24 and can be sent via the interface unit 26 to the external display 30.

With reference to FIG. 3, the tubular body 18 may comprise a plurality of channels 70 for the passage of fluids, connected to respective connectors 34 of the base 16. The channels 70 are open on the front end 48 of the head 42, and extend up to the base 16 parallel to the longitudinal axis of the tubular body 18. The fluid channels 70 can be used to suck secretions and/or to administer drugs or oxygen. The connectors 34 of the base 16 may be connected to suction sources, to pressurized containers for administering oxygen, or they can be connected to devices for administering drugs.

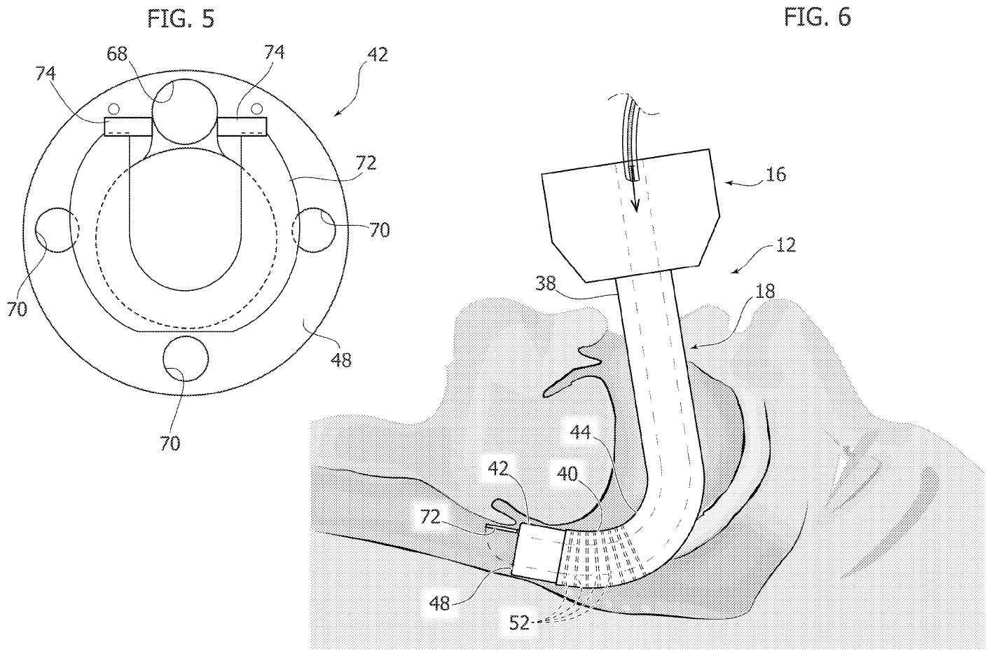

With reference to FIGS. 2 and 5, the laryngoscope 12 may comprise a lifting element of the epiglottis 72, which can be formed of a thin U-shaped plate. The lifting element of the epiglottis 72 is articulated to the front wall 48 of the head 42 by means of two hinges 74, and can be moved between a lowered position (FIG. 5) in which it is in contact with the front wall 48 of the head 42, and a raised position (FIG. 2) in which the element 72 is substantially perpendicular to the frontal surface 48 of the head 42.

With reference to FIG. 3, the tubular body 18 may comprise two Bowden cables 76 arranged to control the movement of the lifting element of the epiglottis between the lowered position and the raised position, and vice versa. The Bowden cables 76 may be connected to a respective actuator 78 (FIG. 1) located in the base 16, which can be controlled by means of the movement control device 28.

With reference to FIG. 1, the base 16 may further comprise another actuator 80 configured to control the insertion of the endotracheal tube 14. For example, the actuator 80 could drive a gear that meshes with a rack formed on the outer surface of the endotracheal tube 14. The actuator 80 may also be controlled by the movement control device 28. Insertion of the endotracheal tube 14 may take place in automatic or semi-automatic mode.

With reference to FIG. 6, the tubular body 18 is inserted into the patient's mouth with the epiglottis lifting element 72 initially in the lowered position. The tubular body 18 is progressively inserted into the larynx, and during insertion the doctor controls the inclination of the head 42 by means of the movement control device 28 to find the correct opening of the trachea. When the device is in the appropriate position to carry out the lifting maneuver of the epiglottis, the actuator 78 is operated which, by tensioning the cables 76, causes lifting of the lifting element of the epiglottis 72.

The images shown on the display 30 allow the doctor to adjust the inclination of the head 42 as necessary to align it with the mouth of the trachea.

When the head 42 is correctly inserted into the trachea, the endotracheal tube 14 is inserted into the tubular body 18. The channel 46 guides the insertion of the endotracheal tube 14. When the endotracheal tube 14 has been inserted into the patient's trachea, the tubular body 18 is extracted leaving the endotracheal tube 14 in place.

In a possible embodiment, the movement control device 28 and the display 30 can be in a remote position with respect to the laryngoscope 12 and can be connected to the electronic control unit 24 of the laryngoscope 12 via the Internet. The images detected by the optical sensor 60 of the laryngoscope 12 are transmitted via the Internet to the display 30, and the doctor can remotely control the movements of the head 42 of the tubular body 18.

The possibility for an expert doctor to remotely control the orientation of the head 42 can enable the insertion of the tubular body 18 of the laryngoscope 12 into a patient's trachea to also be carried out by personnel not qualified to carry out the intubation of a patient. For example, in emergency conditions, first aid personnel may not include a doctor (for example, an anesthesiologist) who is qualified to perform the intubation operation. In this case, the first aid staff could place the laryngoscope into the patient's throat, fix it to the patient's head with a band and a doctor qualified to perform the intubation maneuver could remotely control the most delicate operations that allow the first aid staff aid to correctly intubate a patient.

The tubular body 18 can be made of plastic material with certification for use in medical devices, and can be sanitized during or after the production process. The outer surface of the tubular body 18 may be smooth to reduce the resistance during the insertion step.

The tubular body 18 may be produced in different sizes according to the characteristics of the patient.

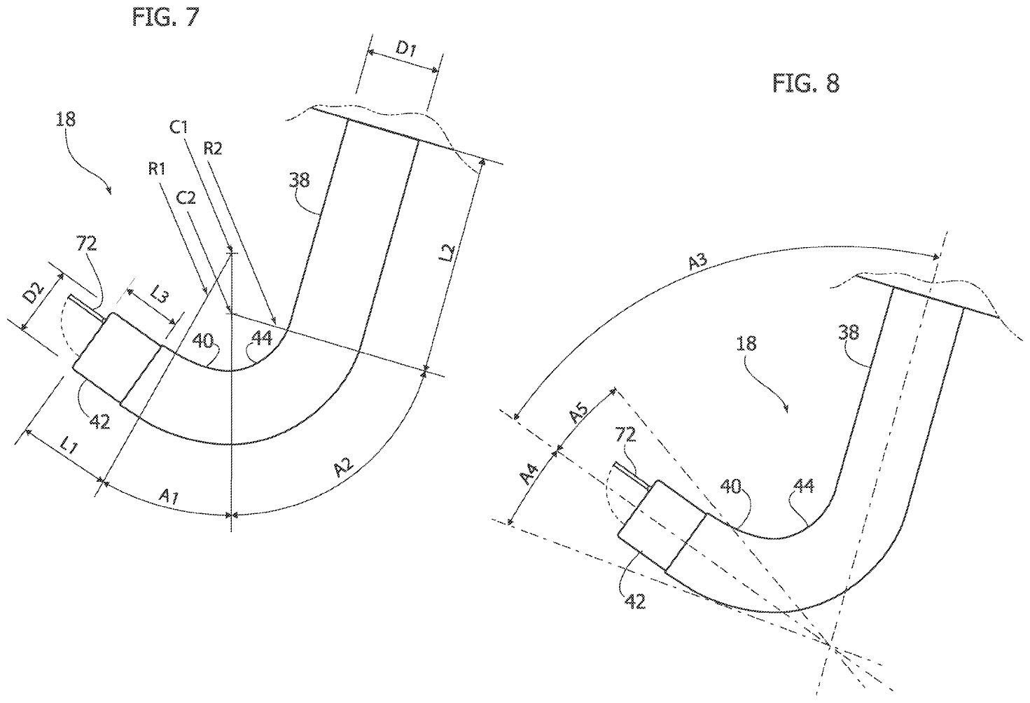

With reference to FIGS. 7-11, the main characteristic measurements of the tubular body 18 can be included in the ranges indicated in the following tables.

TUBULAR BODY—FIGS. 7,8

L1: 15-45 mm straight distal tract length

L2: 55-180 mm proximal portion length

L3: 8-40 mm head length

A1: 0°-43° final part angle

A2: 60°-105° initial part angle

R1: 30-55 mm radius section with angle A1

R2: 35-85 mm radius section with angle A2

D1: 12-40 mm outer diameter of tubular body

D2: 10-40 mm outer diameter of head

A3: 60°-95° body—head axis angle

A4: 0°-30° solid half-angle of head apparatus axis upwards movement

A5: 0°-25° solid half-angle of head apparatus axis downwards movement

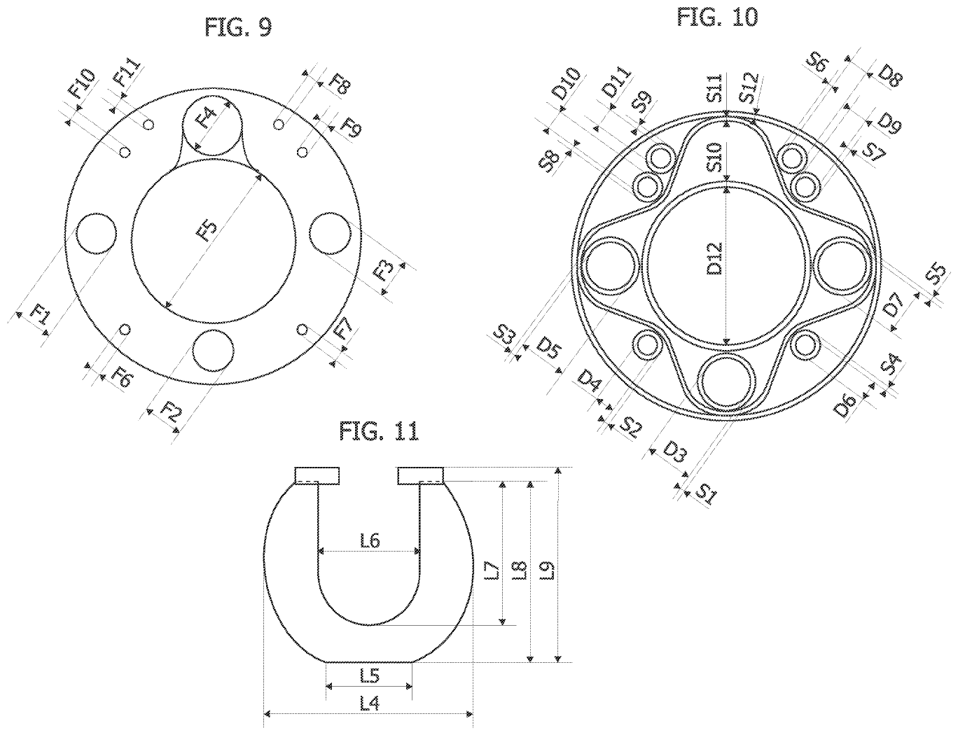

HEAD SECTION—FIG. 9

F1, F2, F3: 0.5-6 mm diameter of service channels holes

F4: 1-8 mm vision system hole diameter

F5: 10-28 mm central channel diameter

F6, F7, F9, F10: 0.5-4 mm diameter of holes for head movement cables

F8, F11: 0.5-2 mm hole diameter for epiglottis lifting cables

BODY SECTION—FIG. 10

D3, D5, D7: 0.5-6 mm diameter of channels for service tubes

S1, S3, S5: 0.1-3 mm thickness of service tubes

D4, D5, D9, D10: 0.8 to 2.6 mm diameter of head movement tubes

S2, S4, S7, S8: 0.1 to 3 mm thickness of head movement tubes

D8, D10: 0.8-2.6 mm diameter of epiglottis lifting movement tubes

S6, S9: 0.1-3 mm thickness of epiglottis lifting movement tubes

D12: 10-28 mm central channel diameter

S10: 0.1-3 mm central channel thickness

S11: 0.5-4 mm outer wall thickness of the tubular body

S12: 0.1-3 mm outer sheath thickness

EPIGLOTTIS LIFTING ELEMENT—FIG. 11

L4: 12-40 mm element width

L5: 0-30 mm width of the lower flat part

L6: 6-24 mm open portion width

L7: 6-24 mm open portion length

L8: 10-38 mm element length

L9: 12-40 mm element length including hinges

Of course, without prejudice to the principle of the invention, the details of construction and the embodiments can be widely varied with respect to those described and illustrated, without thereby departing from the scope of the invention as defined by the claims that follow.

1. A laryngoscope for orotracheal intubation, comprising:
a tubular body having a general J shape, having a central channel configured to guide an endotracheal tube, wherein the tubular bod has a proximal portion, a head and a deformable section that connects the head to the proximal portionpay wherein the deformable section is elastically deformable to vary an orientation of the head with respect to the proximal portion in any plane passing through a longitudinal axis of the head, and
a base connected to one end of the proximal portion opposite to the head, wherein the base comprises a plurality of actuators connected to the head through a transmission system which extends along the tubular body, said plurality of actuators being configured to control the-an inclination angle of said head.

2. The laryngoscope according to claim 1, wherein said transmission system comprises a plurality of Bowden cables, each of which extends from the head to a respective actuator along said tubular body.

3. The laryngoscope according to claim 2, wherein said transmission system comprises four Bowden cables which, in a cross-section, are arranged at vertices of a square whose center coincides with a center of gravity of the tubular body.

4. The laryngoscope according to claim 1, wherein said plurality of actuators are electric actuators controlled by an electronic control unit housed in said base and connected by means of an interface unit to a movement control device located outside said base.

5. The laryngoscope according to claim 4, wherein said movement control device is connected to said electronic control unit through a wireless communication network.

6. The laryngoscope according to claim 1, comprising a vision device arranged in the head and connected to the base by means of a cable extending inside a channel formed within said tubular body.

7. The laryngoscope according to claim 6, wherein an optical signal provided by said vision device is transmitted to a display located outside said base.

8. The laryngoscope according to claim 7, wherein said display is connected to the base via a wireless communication network.

9. The laryngoscope according to claim 1, wherein the head carries a lifting element of an epiglottis, movable between a lowered position and a raised position and connected by transmission cables to an actuator housed in said base.

10. The laryngoscope according to claim 1, comprising a plurality of fluid channels extending inside the tubular body from a front surface of the head at one end of the proximal portion.
